(19) United States
(12) Patent Application Publication
(10) Pub. No.: US 2020/0255808 A1
(43) Pub. Date: Aug. 13, 2020

(54) METHOD FOR PRODUCING A SKIN EQUIVALENT, AND USE THEREOF FOR IN VITRO TESTS AND IN VIVO TRANSPLANTS

(71) Applicant: TECHNISCHE UNIVERSITÄT BERLIN

(72) Inventor: Gerd LINDNER, Berlin (DE)

(73) Assignee: TECHNISCHE UNIVERSITÄT BERLIN, Berlin, OT (DE)

(21) Appl. No.: 15/776,352

(22) PCT Filed: Nov. 14, 2016
(86) PCT No.: PCT/EP2016/077539

§ 371 Date: May 15, 2018

(51) Int. Cl. C12N 5/071; A61L 27/60; A61L 27/38; G01N 33/50

(52) U.S. Cl. CPC C12N 5/0698; A61L 27/60; A61L 27/3804; C12N 2535/10; C12N 2502/092; C12N 2533/54; G01N 33/5088

(57) ABSTRACT

A method for producing a skin equivalent comprising the steps of: a) producing a dermal matrix containing a first layer and a second layer comprises the steps of i) providing the first layer; ii) providing a polymerizable solution comprising a liquid and at least one polymer; iii) adding the polymerizable solution to the first layer; iv) polymerizing the solution to form the second layer; v) compressing the second layer, wherein as a result of the compression the liquid content of the second layer is reduced and the first layer and second layer are joined together to form the dermal matrix; and vi) introducing at least one cavity into the dermal matrix; b) introducing at least one hair follicle and/or hair follicle germ into the at least one cavity of the dermal matrix produced in step a) to produce a skin surrogate; and c) adding at least one further cell population.

The skin is the interface between the body and its environment. It is the first crucial barrier to potentially harmful external influences such as mechanical loads or invasion by pathogenic microorganisms. The essential significance of skin to human health in particular becomes clear when its self-regenerative capacity is no longer sufficient to maintain this protection. This is in particular the case in patients suffering from chronic or extensive acute wounds, as occur in the case of injuries such as burns. If these wounds are not adequately treated with therapies to regenerate the skin, these conditions have a considerable impact on quality of life and may even be fatal.

In addition to the possibilities for regenerating injured skin, artificially produced skin equivalents which inter alia likewise comprise hair follicles may also be used for the therapeutic treatment of the most varied forms of hair loss or alopecia, as it is known.

In addition, there is a need for a suitable test system which can be used for researching the causes of the various types of alopecia. The same applies to investigations into the growth, structure and pigmentation of the hair. In addition to biomedical research, the pharmaceutical and cosmetic industries in particular have an urgent need for suitable systems for testing the efficacy or toxicity of substances on human skin or hair.

Major efforts have been made in recent decades to imitate the complex structures and diverse functions of human skin in the form of a skin replacement. Despite major advances in the development of artificial skin, the still very major differences in structure and function relative to the physiology of natural human skin remain a problem. In particular, the implantation of hair follicles, adipose tissue and vascular supply to artificial skin has hitherto only been unsatisfactorily achieved. Furthermore, the production of such an artificial skin has not hitherto been possible on a large scale since high throughput methods for such products are at present still limited.

The object of the present invention is to reduce or avoid one or more disadvantages of the prior art. In particular, one object of the present invention is to provide a method with which skin equivalents can be produced.

The present invention achieves this object by providing a method for producing a skin equivalent comprising the steps of: a) producing a dermal matrix containing a first layer and a second layer comprising the steps of: i) providing the first layer; ii) providing a polymerizable solution comprising a liquid and at least one polymer selected from collagen, elastin and/or hyaluron; iii) adding the polymerizable solution to the first layer; iv) polymerizing the solution to form the second layer; v) compressing the second layer, wherein as a result of the compression the liquid content of the second layer is reduced and the first layer and second layer are joined together to form the dermal matrix; and vi) introducing at least one cavity into the dermal matrix; b) introducing at least one hair follicle and/or hair follicle germ into the at least one cavity of the dermal matrix produced in step a) to produce a skin surrogate; and c) adding at least one further cell population selected from fibroblasts, endothelial cells, adipocytes, nerve cells, gland cells, melanocytes or keratinocytes to the skin surrogate to produce the skin equivalent.

The production method of the present invention is carried out in vitro. “In vitro” is taken to mean any environment which is not located within a living organism, for example a human or animal body. The in vitro detection method according to the invention thus explicitly does not include a method which is carried out on a human or animal body.

Unless otherwise unambiguously indicated by the context, the use of singular or plural forms always includes both the singular and the plural.

The method according to the invention relates to the production of a skin equivalent. The term “skin equivalent” denotes a skin model which, with regard to nature and function, can be used as a substantially equivalent replacement for natural skin. The skin equivalent preferably has approximately the form and structure of physiological skin.

The method according to the invention for producing a skin equivalent comprises the steps of: a) producing a dermal matrix containing a first layer and a second layer; b) introducing at least one hair follicle and/or hair follicle germ into the at least one cavity of the dermal matrix produced in step a) to produce a skin surrogate; and c) adding at least one further cell population selected from fibroblasts, endothelial cells, adipocytes, nerve cells, gland cells, melanocytes or keratinocytes to the skin surrogate to produce the skin equivalent.

In the method according to the invention for producing a skin equivalent, a dermal matrix is produced in a first step a). Production of the dermal matrix comprises the steps of: i) providing the first layer; ii) providing a polymerizable solution comprising a liquid and at least one polymer selected from collagen, elastin and/or hyaluron; iii) adding the polymerizable solution to the first layer; iv) polymerizing the solution to form the second layer; v) compressing the second layer, wherein as a result of the compression the liquid content of the second layer is reduced and the first layer and second layer are joined together to form the dermal matrix; and vi) introducing at least one cavity into the dermal matrix.

The dermal matrix contains two layers, a first layer and a second layer. In the present case, the “dermal matrix” is taken to mean a material substrate which is used in the method according to the invention as the starting material for the production of the skin equivalent. The dermal matrix here has structural similarities to the layered structure of physiological skin, the first layer accordingly being similar to the (sub)dermal structure and the second layer to the papillary and reticular structure of physiological skin.

In step i), the first layer of the dermal matrix is provided. The first layer of the dermal matrix preferably contains substantially no living cells.

The first layer of the dermal matrix may comprise a natural skin substance. “Skin substance” is taken to mean a material which is similar to or has the connective tissue-like structure of the (sub)dermis of physiological skin. The natural skin substance has preferably been decellularized before being used in the method according to the invention. The natural skin substance may, for example, be donor skin. This donor skin is of animal origin. The donor skin preferably originates from a vertebrate, which may be a mammal, bird, reptile or amphibian, preferably a bird or mammal. This mammal may be a human, monkey, pig, dog, cat or rodent, preferably a human. One donor skin of human origin is for example known in the prior art under the name Epiflex®.

The donor skin may here originate from any suitable region of the body, for example from the trunk, for example from the chest, abdomen or back, or from the limbs, for example from the arms or legs.

Once the skin has been taken from the donor, said skin can be subjected to one or more cleaning steps in order to remove from the skin any interfering substances which may be present. Such a cleaning step may for example include the elimination of living cells.

The first layer of the dermal matrix may comprise an artificial skin substance. The artificial skin substance may, for example, be a material which comprises structural proteins, preferably collagen and/or elastin, particularly preferably collagen and elastin. One artificial skin substance is for example known in the prior art under the name Matriderm®.

The following step ii) involves providing a polymerizable solution. This polymerizable solution is suitable for conversion in a polymerization reaction from the liquid state into a substantially solid state. The polymerizable solution comprises a liquid. The liquid may be, for example, water, a saline solution or a nutrient medium. The liquid may be buffered.

The polymerizable solution preferably has a pH of 2 to 12, preferably of 4 to 10, particularly preferably of 5 to 9, very particularly preferably of 6 to 8.

The polymerizable solution furthermore comprises at least one polymer selected from collagen, elastin and/or hyaluron. The polymerizable solution preferably comprises collagen.

The collagen is preferably a collagen of type I, II, III, IV or V, preferably collagen of type I. The polymerizable solution likewise preferably comprises collagen, elastin and hyaluron.

The at least one polymer is present in the polymerizable solution in a concentration of ≥0.01%, preferably of 0.01% to 50%, particularly preferably 0.1% to 10%, very particularly preferably 0.125% to 5%.

The polymerizable solution may additionally contain cells. These are preferably living cells. The cells may be cells of mesenchymal origin, such as for example multipotent mesenchymal stem cells which are connective tissue precursor cells. The cells are preferably connective tissue cells, particularly preferably fibroblasts.

The following step iii) involves adding the polymerizable solution to the first layer.

In the following step iv), the solution is polymerized to form the second layer of the dermal matrix. This preferably takes place at a temperature of room temperature. “Room temperature” is specified as a temperature in the range from 18° C. to 25° C. The solution is preferably polymerized at a temperature of 25° C., preferably at 30° C., particularly preferably at 30° C. to 50° C., very particularly preferably at 35° C. to 45° C., in particular preferably at 37° C. to 42° C.

Polymerization of the solution to form the second layer may take place under substantially static conditions or with agitation. Polymerization preferably takes place with rotating, swiveling or shaking agitation. Polymerization of the solution to form the second layer may likewise take place under conditions which combine both of the methods just stated, such that the polymerization reaction is for example firstly initiated with agitation and then taken to completion under substantially static conditions.

Polymerization of the solution to form the second layer takes place for at least 1 min, preferably for 2 min to 24 h, particularly preferably for 5 min to 12 h, particularly preferably 10 min to 4 h, very particularly preferably for 15 min to 1 h.

Prior to step iv), the polymer in the polymerizable solution can optionally be patterned by agitation. Patterning of the polymer brings about a uniform distribution of the polymer in the solution, such that the polymerized second layer created therefrom has a substantially uniform structure.

The following step v) involves compressing the second layer, wherein as a result of the compression the liquid content of the second layer is reduced and the first layer and second layer are joined together to form the dermal matrix. Compression of the second layer may here proceed by centrifugation, sedimentation or mechanical application of force. The mechanical application of force is preferably pressing. A proportion of the liquid content which is removed from the second layer by compression may here be absorbed by the first layer.

As a result of the compression, the first and second layers join together to form the dermal matrix. The dermal matrix has a layer thickness of at least 0.1 mm, preferably 0.25 mm to 10 mm, particularly preferably 0.5 mm to 5 mm, very particularly preferably 1 mm to 3 mm. The first layer and the second layer within the dermal matrix may here each have the same or a different layer thickness. The first layer has a layer thickness of at least 0.01 mm, preferably 0.05 mm to 5 mm, particularly preferably 0.1 mm to 2.5 mm. The second layer has a layer thickness of at least 0.01 mm, preferably 0.05 mm to 5 mm, particularly preferably 0.1 mm to 2.5 mm.

In the next step vi), at least one cavity is introduced into the dermal matrix. The cavities of the dermal matrix have an average diameter of 0.001 mm to 5 mm, preferably of 0.005 mm to 1 mm, particularly preferably of 0.01 mm to 0.08 mm, very particularly preferably of 0.02 mm to 0.06 mm, in particular preferably of 0.05 mm to 0.04 mm. The cavities of the dermal matrix have a depth of 0.05 mm to 10 mm, preferably of 0.1 mm to 8 mm, particularly preferably of 0.15 mm to 6 mm, very particularly preferably of 0.2 mm to 4 mm, in particular preferably of 0.5 mm to 2.5 mm.

Preferably, 1 to 400 cavities/cm² are introduced into the dermal matrix, preferably 10 to 200/cm², particularly preferably 20 to 100/cm², very particularly preferably 30 to 100/cm². The cavities may here be introduced into the dermal matrix in a regular or irregular spacing; the cavities are preferably introduced into the dermal matrix in a regular spacing.

The at least one cavity may be introduced by removing material from the dermal matrix. The at least one cavity is preferably introduced into the dermal matrix by piercing, excision or by means of an impression mold. Piercing proceeds, for example, with the aid of a needle, in particular a cannula. Excision may, for example, proceed by means of a scalpel, optionally with the assistance of a further tool such as for example a pair of forceps in order to remove the excised material from the dermal matrix.

Alternatively, the at least one cavity may be introduced into the dermal matrix by means of an impression mold. The impression mold comprises a base. The base may have various shapes, for instance it may for example have a round, oval or polygonal base area. The base may be planar or patterned. The impression mold preferably comprises at least one cylinder. The term “cylinder” is taken to mean an elongate cylindrical body which is suitable for introducing a cavity into the dermal matrix. The at least one cylinder has a diameter of ≥0.001 mm to 5 mm, preferably of 0.005 mm to 1 mm, particularly preferably of 0.01 mm to 0.08 mm, very particularly preferably of 0.02 mm to 0.06 mm, in particular preferably of 0.05 mm to 0.04 mm. The at least one cylinder has a length of 0.05 mm to 10 mm, preferably of 0.1 mm to 8 mm, particularly preferably of 0.15 mm to 6 mm, very particularly preferably of 0.2 mm to 4 mm, in particular preferably of 0.5 mm to 2.5 mm.

The at least one cylinder is preferably fastened to the base; the at least one cylinder is particularly preferably integrated in the base. The at least one cylinder is preferably oriented perpendicular to the base area of the base.

The impression mold preferably comprises cylinder. The impression mold preferably comprises 10 to 10000 cylinders, particularly preferably 20 to 5000, very particularly preferably 100 to 1000. The cylinders may be present in the impression mold in a regular or irregular spacing. The cylinders are preferably present in the impression mold in a regular spacing.

The at least one cylinder preferably at least in part comprises a hollow space; the cylinder is particularly preferably substantially completely hollow.

The at least one cylinder may have a funnel-shaped flared portion at one of the ends thereof. In this case, the cylinder is preferably substantially completely hollow. The funnel-shaped flared portion of the cylinder has, at the outer end thereof, a diameter of 0.01 mm, preferably of 0.1 mm to 4 mm, particularly preferably of 0.2 mm to 3 mm, very particularly preferably of 0.3 mm to 2.5 mm, in particular preferably of 0.5 mm to 2 mm.

The impression mold preferably likewise comprises at least one raised portion. The expression “raised portion” denotes an elevation which is substantially conical in shape. The raised portion preferably has the shape of a right cone, wherein the tip of the cone is preferably rounded. A wave-like pattern is impressed into the dermal matrix with the aid of said at least one raised portion, which structure is similar to the physiological papillary structure of natural skin at the epidermal/dermal boundary. The base area of the conical raised portion has a diameter of 0.01 mm, preferably of 0.1 mm to 4 mm, particularly preferably of 0.2 mm to 3 mm, very particularly preferably of 0.3 mm to 2.5 mm, in particular preferably of 0.5 mm to 2 mm. The rounded tip of the cone has a diameter of 0.001 mm, preferably of 0.01 mm to 3.5 mm, particularly preferably of 0.1 mm to 2.5 mm, very particularly preferably of 0.2 mm to 2 mm, in particular preferably of 0.3 mm to 1 mm.

The at least one raised portion is preferably fastened to the base; the at least one raised portion is particularly preferably integrated in the base. It is likewise possible for the at least one raised portion to be a patterning unit which imparts a corresponding shape to a patterned base. The height of the at least one raised portion is preferably oriented perpendicular to the base area of the base.

The impression mold preferably comprises raised portion. The impression mold preferably comprises 10 to 10000 raised portions, particularly preferably 20 to 5000, very particularly preferably 100 to 1000. The raised portions may be present in the impression mold in a regular or irregular spacing. The raised portions are preferably present in the impression mold in a regular spacing.

The impression mold preferably comprises at least one cylinder and at least one raised portion. The impression mold preferably comprises 10 to 10000 cylinders and raised portions, particularly preferably 20 to 5000, very particularly preferably 100 to 1000. The cylinders and/or raised portions may in each case be present in the impression mold in a regular or irregular spacing. The cylinders and/or raised portions are preferably present in the impression mold in a regular spacing; the cylinders and raised portions are preferably present in the impression mold in a regular spacing. The cylinders and raised portions are preferably present in the impression mold in such a manner that they are substantially offset from one another.

The base of the impression mold, the at least one cylinder and/or the at least one raised portion may for example consist of metal, plastics, rubber or silicone such as for example polydimethylsiloxane (PDMS) or comprise these materials. The base of the impression mold, the at least one cylinder and the at least one raised portion preferably consist of the same material. The base, the at least one cylinder and the at least one raised portion particularly preferably substantially take the form of a one-piece impression mold.

Method steps iv), v) or vi) for producing the dermal matrix may take place in succession and/or simultaneously. Introduction of the at least one cavity into the dermal matrix in step vi) preferably proceeds during or after the polymerization in step iv). Steps iv) and vi) particularly preferably proceed simultaneously, such that the at least one cavity is preferably introduced into the dermal matrix during polymerization. Steps iv) and vi) in particular preferably proceed simultaneously, wherein polymerization preferably takes place with agitation, particularly preferably with rotating, swiveling or shaking agitation.

Once the dermal matrix has been produced in step a), at least one hair follicle and/or hair follicle germ is introduced into the at least one cavity of the dermal matrix in step b) of the method according to the invention to produce a skin surrogate. One, a plurality or all of the cavities in the dermal matrix is/are preferably coated with extracellular matrix proteins prior to introduction of the hair follicle and/or hair follicle germ in step b).

The term “hair follicle” denotes natural structures which surround the hair root and so anchor the hair in the skin. In cross-section, the hair follicle comprises two layers, namely an outer and an inner epithelial hair root sheath. The outer epithelial hair root sheath is a funnel-like invagination on the basal layer of the skin which forms an envelope for the hair root. The inner epithelial hair root sheath comprises the hair root and consists of three layers, namely an inner layer (cuticle), a middle layer (Huxley's layer) and an outer layer (Henle's layer). Glands which produce and secrete sebum and other substances such as for example scents open into the hair follicle. In the vicinity of the sebaceous glands, hair erector muscles, or arrector pili muscles, which are capable of making the hair stand on end and are responsible for “goose pimples”, are likewise located on the hair follicle. Fine nerve fibers used for the sense of touch and to control the hair erector muscles furthermore end in the hair follicle. According to the invention, the term “hair follicle” likewise includes a microfollicle structure which is incomplete in comparison with natural microfollicles, which structure may include the outer and/or inner epithelial hair root sheath, but without containing further cell types such as muscle or nerve cells, blood vessels etc., such that the hair follicle is smaller in size in comparison with natural hair follicles. A hair follicle has a three-dimensional, optionally spatially delimited shape which is similar to the three-dimensional appearance of a physiological hair follicle. The hair follicles may have a polar structure, for example a pointed projection on one side of the hair follicle, which is reminiscent of the structures in early hair morphogenesis.

The hair follicle used may thus be a hair follicle isolated from tissue. Methods for removing hair follicles from tissue are known to a person skilled in the art. The hair follicles are preferably removed from the tissue in accordance with good medical practice. The hair follicles are preferably removed from the tissue in such a way that they remain substantially intact, i.e. undamaged. The hair follicles can for example be removed from the tissue as follows by: i) separating the epidermis from the underlying dermis or adipose tissue in the tissue, preferably using a scalpel; ii) dissecting out hairs from the dermis or the adipose tissue of the tissue. The removed hairs comprise the hair follicles at their proximal ends. Prior to step ii), the dermis or the adipose tissue may optionally be compressed to facilitate subsequent dissecting out of the hair follicles present therein. Compression may here for example be carried out with the aid of a pair of forceps. The hair follicles are preferably removed under a microscope.

The tissue from which the hair follicles may be obtained originates from a mammal. This mammal may be a human, monkey, pig, dog, cat or rodent, preferably a human. The mammal is thus a donor for the tissue containing the hair follicles.

The tissue preferably comprises skin. The tissue may here be obtained from any hairy region of the body, preferably from the head, eyebrows, beard, genital area, leg or other regions of the body. The tissue is preferably obtained by means of a biopsy.

Before the hair follicles are removed from the tissue, said tissue may be subjected to one or more cleaning steps in order to remove from the tissue any interfering substances which may be present.

Allogeneic hair follicles are preferably used for the skin equivalent, i.e. the donor of the tissue from which the hair follicles are isolated, and the recipient of the skin equivalent which comprises the isolated hair follicles belong to the same species and thus for example both donor and recipient are human. It is particularly preferred to use autologous hair follicles for the skin equivalent, i.e. for the donor of the tissue from which the hair follicles are isolated and the recipient of the skin equivalent which comprises the isolated hair follicles to be the same person, for example the same human.

It is likewise possible for hair follicle formation to proceed in the cavities of the dermal matrix. To this end, at least one hair follicle germ is introduced into the at least one cavity of the dermal matrix in step b) of the method according to the invention.

The term “hair follicle germ” denotes an early stage of a hair follicle from which said hair follicle develops. The hair follicle germ is preferably at least half the size of a physiological dermal papilla (DP) of a hair follicle after isolation thereof. The hair follicle germ likewise preferably has roughly the shape of a physiological DP of a hair follicle after isolation thereof. The hair follicle germs consist of approx. 2 to 20000 cells, preferably of 10 to 15000, particularly preferably of 1000 to 10000, very particularly preferably of 1500 to 5000 cells.

The hair follicle germ comprises dermal papilla fibroblasts (DPF). The hair follicle germ may furthermore contain connective tissue sheath fibroblasts (CTSF). The hair follicle germ optionally comprises at least one further cell population selected from endothelial cells, cells of stromal vascular fractions (SVF), keratinocytes (KC), melanocytes (MC) or fibroblasts, such as for example CTSF.

The DPF for the hair follicle germ are isolated from at least one DP, wherein the DP originates from at least one hair follicle.

Methods for obtaining the DP or DPF are known to a person skilled in the art and described for example in documents EP 2 274 419 B1 and DE 101 62 814 B4. The DP can be provided from the hair follicle and the DPF isolated from the DP for example as follows: isolated hair follicles are immobilized on the hair shaft, for example with a pair of forceps, and the connective tissue sheath is separated diametrically from the hair shaft, for example with another pair of forceps, so everting the bulb and thus exposing the DPF and the hair shaft with the hair matrix. In this way, the DP can be separated from the remaining part of the hair follicle, which may be achieved for example with the aid of a needle or cannula.

The isolated DP are then for example transferred into a cell culture vessel and mechanically immobilized on the surface of the cell culture vessel. The DPF are preferably obtained in that the isolated DP are mechanically held down on the bottom of the cell culture vessel, for example by means of a needle tip or a scalpel. The morphology of the DP is largely retained here, but the basal lamina of the DP is slightly perforated, such that the DPF can migrate out of the DP. The DPF can also be obtained from the DP with the aid of enzymatic separation.

After being isolated and prior to being used in the method according to the invention, the DPF may be subjected to intermediate culturing, for example to multiply their number. The DPF used in the method according to the invention may accordingly be descendants or clones of the originally isolated DPF.

The hair follicles from which the DP and DPF are isolated originate from tissue. Methods for removing hair follicles from tissue are known to a person skilled in the art. The hair follicles are preferably removed from the tissue in accordance with good medical practice. The hair follicles are preferably removed from the tissue in such a way that they remain substantially intact, i.e. undamaged. The hair follicles can for example be removed from the tissue as follows by: i) separating the epidermis from the underlying dermis or adipose tissue in the tissue, preferably using a scalpel; ii) dissecting out hairs from the dermis or the adipose tissue of the tissue. The removed hairs comprise the hair follicles at their proximal ends. Prior to step ii), the dermis or the adipose tissue may optionally be compressed to facilitate subsequent dissecting out of the hair follicles present therein. Compression may here for example be carried out with the aid of a pair of forceps. The hair follicles are preferably removed under a microscope.

The tissue from which the hair follicles may be obtained, for example for isolating the DP or DPF, originates from a mammal. This mammal may be a human, monkey, pig, dog, cat or rodent, preferably a human. The mammal is thus a donor for the tissue containing the hair follicles. The mammal is particularly preferably a human patient suffering from hair loss.

The tissue preferably comprises skin. The tissue may here be obtained from any hairy region of the body, preferably from the head, eyebrows, beard, genital area, leg or other regions of the body. The tissue is preferably obtained by means of a biopsy.

Before the hair follicles are removed from the tissue, for example for isolating the DP or DPF, said tissue may be subjected to one or more cleaning steps in order to remove from the tissue any interfering substances which may be present.

In addition to the DPF, the hair follicle germ may also contain connective tissue sheath fibroblasts (CTSF). The CTSF for the hair follicle germ originate from at least one hair follicle.

The CTSF can be isolated from the hair follicles for example as follows: isolated hair follicles are immobilized on the hair shaft, for example with a pair of forceps, and the connective tissue sheath is separated diametrically from the hair shaft, for example with another pair of forceps, so everting the bulb. In this way, the proximal part of the bulb with the CTSF can be separated from the remaining part of the hair follicle, which may be achieved for example with the aid of a needle or cannula.

After being isolated and prior to being used in the method according to the invention, the CTSF may be subjected to intermediate culturing, for example to multiply their number. The CTSF used in the method according to the invention may accordingly be descendants or clones of the originally isolated DPF.

The hair follicles from which the CTSF are isolated originate from tissue. Methods for removing hair follicles from tissue are known to a person skilled in the art. The hair follicles are preferably removed from the tissue in accordance with good medical practice. The hair follicles are preferably removed from the tissue in such a way that they remain substantially intact, i.e. undamaged. The hair follicles can for example be removed from the tissue as follows by: i) separating the epidermis from the underlying dermis or adipose tissue in the tissue, preferably using a scalpel; ii) dissecting out hairs from the dermis or the adipose tissue of the tissue. The removed hairs comprise the hair follicles at their proximal ends. Prior to step ii), the dermis or the adipose tissue may optionally be compressed to facilitate subsequent dissecting out of the hair follicles present therein. Compression may here for example be carried out with the aid of a pair of forceps. The hair follicles are preferably removed under a microscope.

The tissue from which the hair follicles may be obtained, for example for isolating the CTSF, originates from a mammal. This mammal may be a human, monkey, pig, dog, cat or rodent, preferably a human. The mammal is thus a donor for the tissue containing the hair follicles. The mammal is particularly preferably a human patient suffering from hair loss.

The tissue preferably comprises skin. The tissue may here be obtained from any hairy region of the body, preferably from the head, eyebrows, beard, genital area, leg or other regions of the body. The tissue is preferably obtained by means of a biopsy.

Before the hair follicles are removed from the tissue, for example for isolating the CTSF, said tissue may be subjected to one or more cleaning steps in order to remove from the tissue any interfering substances which may be present.

The DPF and CTSF are thus obtained from isolated hair follicles. The DPF and CTSF may here be obtained from the same hair follicle. The DPF and CTSF may, however, also be obtained from different hair follicles. If the DPF and CTSF are obtained from different hair follicles, these hair follicles may have been isolated from the same tissue, for example from tissue which was for example taken from the nape region of a donors head. If the DPF and CTSF are obtained from different hair follicles, these hair follicles may also have been isolated from different tissues, such that for example one of the tissues was for example taken from the nape region of a donors head while the other tissue was for example taken from a donors beard. In the latter case, the donor may be the same or a different donor. If it is a different donor, said donor is preferably an allogeneic donor. It is preferably the same, i.e. autologous, donor.

In addition to the DPF and optionally the CTSF, the hair follicle germ may also contain at least one further cell population selected from endothelial cells, cells of stromal vascular fractions (SVF), keratinocytes (KC), melanocytes (MC) or fibroblasts. The endothelial cells (EC) optionally present in the hair follicle germ may be isolated with the aid of methods known in the prior art for obtaining EC.

The EC may be obtained from lymph or blood vessels, preferably from lymph or blood vessels located in the vicinity of hair follicles. The EC are preferably obtained from a human donor. The EC are particularly preferably autologous. After being isolated and prior to being used in the method according to the invention, the EC may be subjected to intermediate culturing, for example to multiply their number. The EC used in the method according to the invention may accordingly be descendants or clones of the originally isolated EC.

The hair follicle germ may comprise cells of stromal vascular fractions (SVF). The SVF comprise various cell populations such as precursors of adipocytes (preadipocytes), endothelial cells, endothelial muscle cells, fibroblasts, macrophages or blood cells. The SVF may for example be obtained by excising adipose tissue or by liposuction. Methods for obtaining the SVF are known to a person skilled in the art. The isolation of SVF from adipose tissue is described for example in document WO 2014 036 094 A1. The SVF are preferably obtained from a human donor, the SVF particularly preferably being autologous. After obtaining the SVF from the donor, specific components of the SVF can be removed from the SVF, which removal may proceed by one or more washing, cleaning or isolation steps or also by targeted separation or removal of the components. After being isolated and prior to being used in the method according to the invention, the SVF may likewise be subjected to intermediate culturing, for example to multiply their number. The SVF used in the method according to the invention may accordingly be descendants or clones of the originally isolated SVF. Thanks to the presence of the SVF, elevated or faster cell multiplication of the DPF and CTSF is achieved and formation of the hair follicle germs is accordingly accelerated.

The hair follicle germ may furthermore comprise keratinocytes (KC), melanocytes (MC) and/or CTSF. The KC, MC and/or CTSF need not originate from a hair follicle, but may instead be obtained from other mammalian tissues, such as for example skin. Methods for obtaining the KC, MC and CTSF are known to a person skilled in the art and are described for example in document DE 101 62 814 B4 and in Toma et al., Stem cells 23, 727-37 (2005), Barrandon and Green, Proc. Natl. Acad. Sci. 82, 5390-4 (1985), Tobin et al., J. Invest. Dermatology 104(1), 86-88 (1995) and Magerl et al., Method Exp. Dermat. 11, 381-385 (2002). The at least one further cell population may preferably be obtained from a hair follicle and/or is obtained from a hair follicle.

The KC, MC and/or CTSF may be obtained from the same hair follicle. The KC, MC and/or CTSF may, however, also be obtained from different hair follicles. If the KC, MC and/or CTSF are obtained from different hair follicles, these hair follicles may have been isolated from the same tissue, for example from tissue which was for example taken from the nape region of a donor's head. If the KC, MC and/or CTSF are obtained from different hair follicles, these hair follicles may also have been isolated from different tissues, such that for example one of the tissues was for example taken from the nape region of a donor's head while another tissue was for example taken from a donor's beard. In the latter case, the donor may be the same or a different donor. If it is a different donor, said donor is preferably an allogeneic donor. It is preferably the same, i.e. autologous, donor.

The at least one further cell population is particularly preferably obtained from the same hair follicle from which the DP was isolated. After being isolated and prior to being used in the method according to the invention for producing the hair follicles, the KC, MC and/or CTSF may be subjected to intermediate culturing, for example to multiply their number. The KC, MC and/or CTSF used in the method according to the invention may accordingly be descendants or clones of the originally isolated KC, MC and/or CTSF.

The hair follicle germ preferably comprises DPF and CTSF. The hair follicle germ particularly preferably comprises DPF, CTSF and SVF. The hair follicle germ very particularly preferably comprises DPF, CTSF, SVF, KC and MC.

The hair follicle germ may comprise a coating which contains one or more different extracellular matrix proteins such as for example collagen IV, fibronectin and/or laminin. This coating may be created by the DPF or CTSF themselves while they are forming the hair follicle germ. Alternatively or additionally thereto, the hair follicle germ may also be coated with the one or more different extracellular matrix proteins once it has been formed.

If a hair follicle germ is introduced into the dermal matrix in step b) of the method according to the invention, said step b) may for example be carried out as follows: the cavities of the dermal matrix are preferably firstly coated with extracellular matrix proteins and then colonized with DPF and/or CTSF. Preferably, it is the CTSF which are introduced first into the cavities and then the DPF. EC and/or SVF may optionally then be introduced into the cavities. MC and/or KC, preferably MC and KC, are added in a subsequent step.

Introduction of the at least one hair follicle and/or hair follicle germ into the dermal matrix gives rise to a skin surrogate. According to the invention, a “skin surrogate” is taken to mean a skin replacement which is not fully adequate. The skin surrogate is taken to be an intermediate from which the skin equivalent is produced.

After step b), the skin surrogate is preferably incubated for at least 10 min before step c) is carried out. Incubation is preferably carried out for 20 min to 48 h, particularly preferably for 30 min to 24 h, particularly preferably for 1 h to 12 h.

The following step c) of the method according to the invention involves adding at least one further cell population selected from fibroblasts, endothelial cells, adipocytes, nerve cells, gland cells, melanocytes or keratinocytes to the skin surrogate in order to produce the skin equivalent. Methods for obtaining the adipocytes, nerve cells or gland cells are known to a person skilled in the art.

After step c) of the method according to the invention, the skin equivalent is incubated for at least 10 min before the skin equivalent is put to further use, for example as a transplant or for testing substances. Incubation is preferably carried out for at least 20 min, particularly preferably for 1 h to 21 days, particularly preferably for 5 to 15 days, very particularly preferably for 8 to 12 days.

The present invention also relates to a skin equivalent which is produced by the method according to the invention.

The present invention moreover comprises a transplant which comprises an effective amount of the skin equivalent optionally together with pharmaceutically acceptable adjuvants.

One embodiment of the method for producing the skin equivalent comprises the steps of: a) producing a dermal matrix containing a first layer and a second layer comprising the steps of: i) providing the first layer; ii) providing a polymerizable solution comprising a liquid and at least one polymer selected from collagen, elastin and/or hyaluron; iii) adding the polymerizable solution to the first layer; iv) polymerizing the solution to form the second layer; v) compressing the second layer, wherein as a result of the compression the liquid content of the second layer is reduced and the first layer and second layer are joined together to form the dermal matrix; and vi) introducing at least one cavity into the dermal matrix; b) introducing at least one hair follicle and/or hair follicle germ into the at least one cavity of the dermal matrix produced in step a) to produce a skin surrogate; and c) adding at least one further cell population selected from fibroblasts, endothelial cells, adipocytes, nerve cells, gland cells, melanocytes or keratinocytes to the skin surrogate to produce the skin equivalent.

The first layer is preferably an artificial skin substance; the artificial skin substance particularly preferably comprises collagen and/or elastin, in particular preferably collagen and elastin.

The polymerizable solution comprises collagen, preferably type I collagen, as the polymer. The polymerizable solution particularly preferably additionally comprises connective tissue cells, preferably fibroblasts.

Prior to step iv), the polymer in the polymerizable solution is preferably patterned by agitation.

Polymerization of the solution to form the second layer in step iv) and introduction of at least one cavity into the dermal matrix in step vi) preferably proceed simultaneously. Step iv) and step vi) particularly preferably proceed simultaneously and with agitation, particularly preferably with rotating agitation.

Compression of the second layer in step v) preferably proceeds by centrifugation.

In step b), at least one hair follicle germ is preferably introduced into the at least one cavity of the dermal matrix, wherein the cavity is particularly preferably coated with extracellular matrix proteins, before firstly CTSF and then DPF are introduced into the cavity. EC and/or SVF are preferably additionally introduced into the cavity. MC and/or KC, preferably MC and KC, are then added to the cavities.

In step c), EC, adipocytes, MC and/or KC, preferably EC, adipocytes, MC and KC, are added to the skin surrogate generated in step b). Nerve cells and gland cells are particularly preferably additionally added to the skin surrogate.

The present invention also relates to use of the skin equivalent which has been produced by the method according to the invention, and/or of the transplant comprising the skin equivalent for therapeutic skin regeneration or for the treatment of a reduced quantity of skin. It is here preferable to use allogeneic cells for producing the skin equivalent, which means that the donor of the cells used and the recipient of the produced skin equivalent belong to the same species, i.e. that both donor and recipient are for example human. It is particularly preferred for the therapeutic treatment of a condition involving a reduced quantity of skin to produce the skin equivalent using autologous cells, which means that the donor of the cells used and the recipient of the produced skin equivalent are identical, i.e. for example the same human being.

The present invention also relates moreover to the use of the skin equivalent according to the invention for in vitro testing of substances for cosmetic, pharmacological or toxic characteristics.

The present invention furthermore relates to the use of the skin equivalent according to the invention for in vitro testing of skin- and/or hair-regulating substances.

The present invention also comprises a kit for carrying out the method according to the invention. To this end, the kit may comprise the skin equivalent and/or the transplant according to the invention, in order for example to carry out the methods according to the invention for therapeutic skin regeneration or the treatment of a condition involving a reduced quantity of skin or substance screening. The kit may optionally also include instructions for use of the kit and/or for carrying out the method according to the invention. The kit may furthermore comprise further components for carrying out the method according to the invention, such as for example reaction vessels, filters, solutions and/or other agents.

The present invention also relates to a method for in vitro screening of substances with skin- and/or hair-regulating characteristics which comprises the following steps: a) providing a sample of the skin equivalent; b) dividing the corresponding sample into portions; c) incubating at least one portion with substances which are to be tested; and d) comparing the parameters of the skin and/or hair characteristics in the portion with another portion which has not been incubated with the substances.

The present invention also comprises a test system comprising the skin equivalent produced by the method according to the invention for the in vitro investigation of the causes of hair loss caused by illness.

The invention is explained in greater detail below with reference to exemplary embodiments and the associated figures.

### FIGURES

FIG. 1 Photographic image of a dermal matrix.

FIG. 2 Photographic image of a skin equivalent, a hair follicle and hair follicle germs.

FIG. 3 Photographic image of an impression mold for introducing cavities into a dermal matrix.

FIG. 4 Schematic diagram of an impression mold for introducing cavities into a dermal matrix. A: sectional view, B: plan view.

FIG. 5 Schematic diagram of an impression mold for introducing cavities into a dermal matrix. A: plan view of the top of the impression mold, B: plan view of the bottom of the impression mold, C and D: side view.

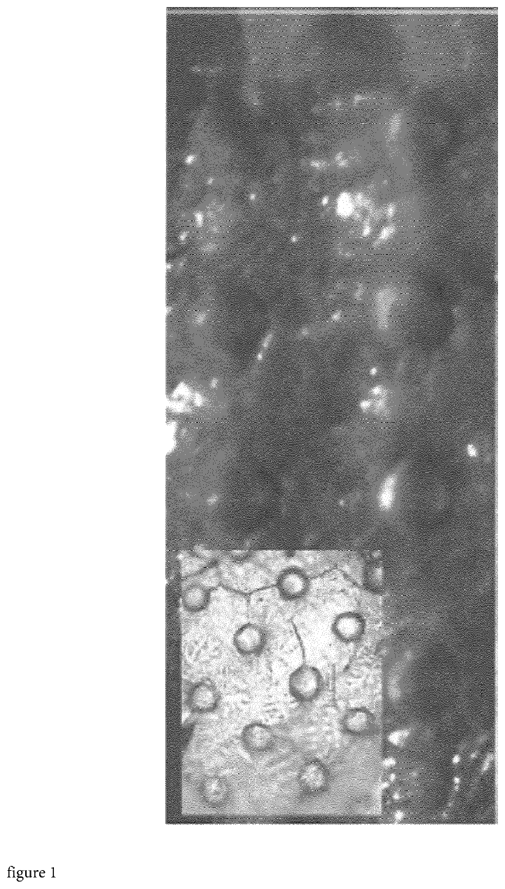

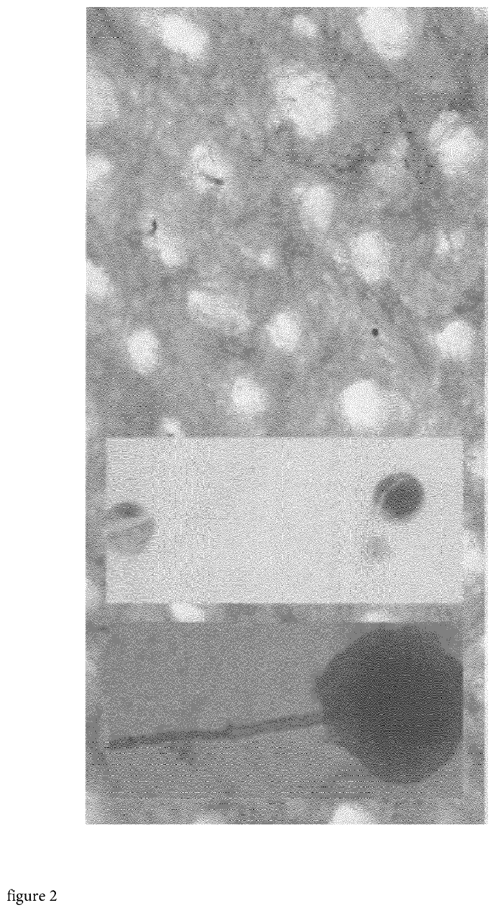

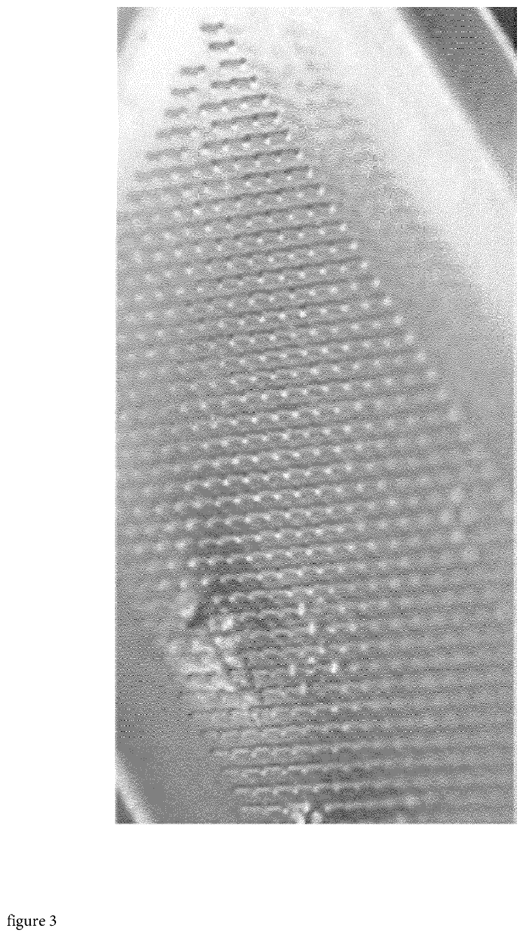

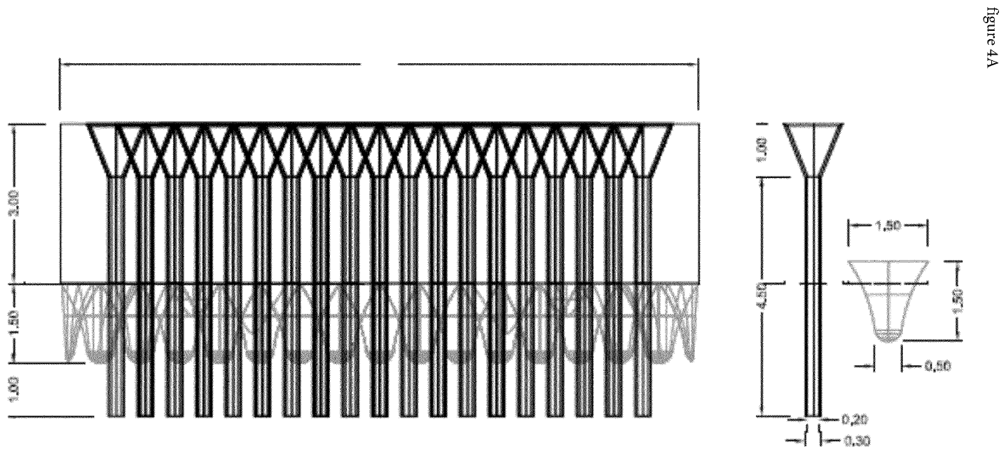

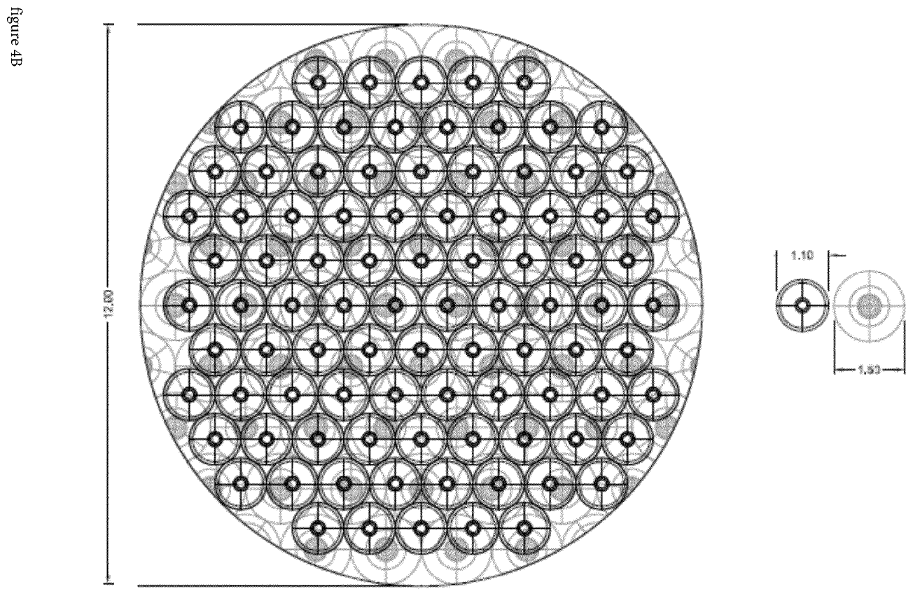

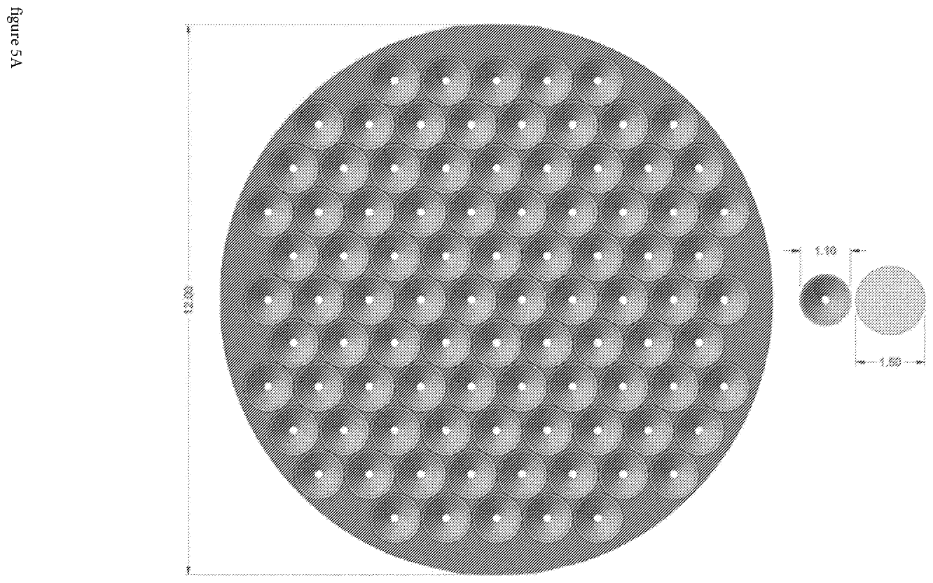

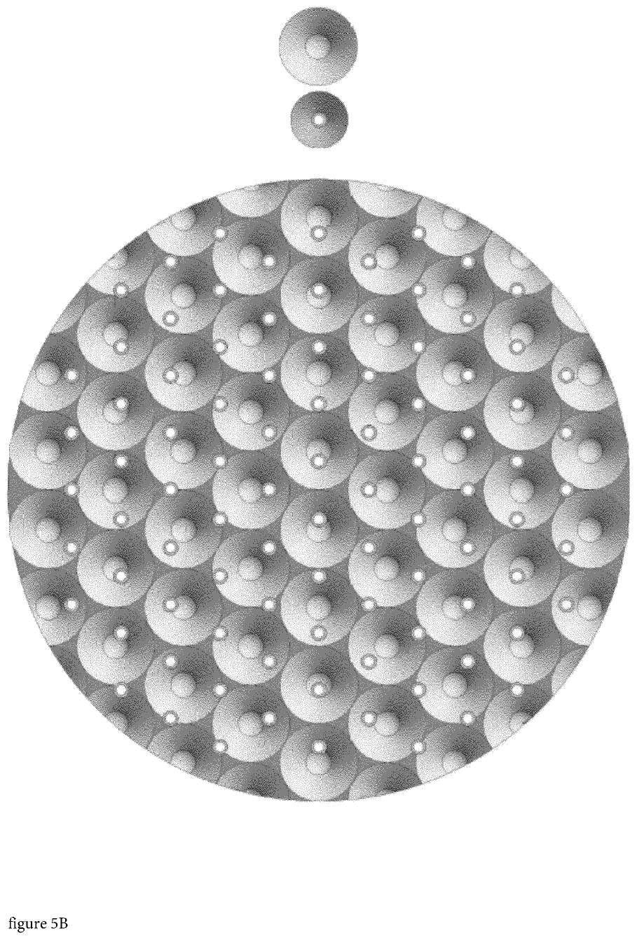

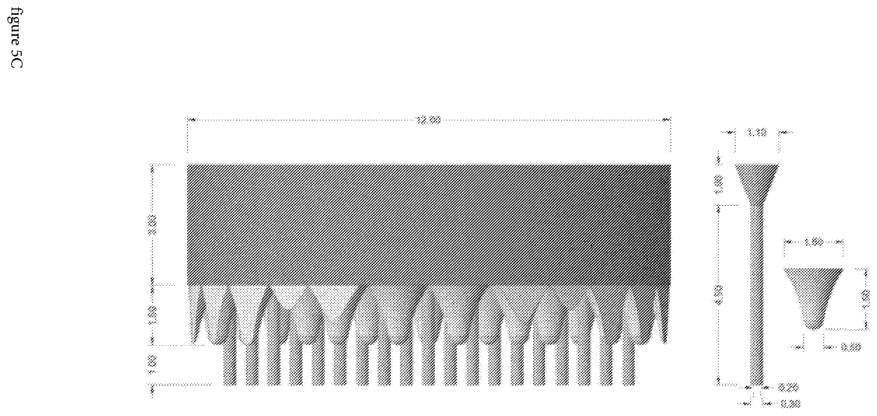

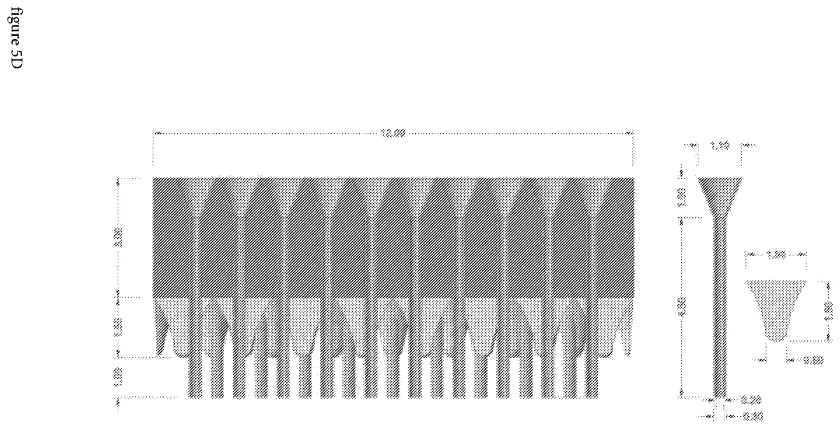

### EXAMPLES

Isolation of the Various Cell Types from Hair Follicles

DPF, CTSF, MC and KC were isolated from the human hair follicle in accordance with a modified form of the standard protocols (described for example in “Mager) et al.”). Isolated hair follicles from punch biopsies or dissected hair follicles from an FUT hair transplantation were immobilized on the hair shaft with a pair of forceps and the connective tissue sheath was incised and diametrically separated therefrom carefully with another pair of forceps, so everting the bulb and exposing the dermal papilla and the hair shaft with the hair matrix. In this manner, the proximal part of the bulb with the connective tissue sheath fibroblasts and the dermal papilla could very easily be separated from the remainder of the hair follicle with the assistance of a needle/cannula. The hair shaft, which contains the hair matrix keratinocytes and melanocytes which are likewise required, was also optimally dissected for further culturing.

The dermal papillae and connective tissue envelopes extracted in this manner from the hair follicles which were taken were each collected in a separate vessel comprising medium. The DPF and CTSF were dissolved out and isolated from the surrounding tissue by gentle tissue dissociation using a tissue dissociator and the associated extraction kit (for example gentleMACS Dissociator #130-093-235, whole skin dissociation kit #130-101-540, Miltenyi Biotec). To this end, the isolated tissue fragments (dermal papilla, connective tissue envelope and hair shaft with epithelial/neuroectodermal cells), 435 μl buffer L, and an enzyme mix of 12.5 mg enzyme P and/or 4.50 mg enzyme D and/or 2.5 mg enzyme A were each introduced into a gentleMACS C tube and carefully mixed.

The sample was incubated in a water bath at 37° C. for 1-3 hours or overnight, longer incubation times increasing cell yield. After incubation, the sample was diluted by adding 0.5 ml cold cell culture medium. The C tube was sealed and fastened upside down onto the sleeve of the gentleMACS dissociator. The “h_skin_01” program was then run. Once the program was complete, a short centrifugation step was then carried out to collect the test material on the bottom. The cells could be washed with fresh medium and the cell suspension separated by a 70 μm filter. It is possible to dispense with the above-described addition of enzymes, which are undesirable in the case of direct autologous therapy, with the assistance of further dissociation runs, although in this case lower cell yields must be expected.

Production of Cells from a Stromal Vascular Fraction (SVF)

A 1 liter fraction from tumescent liposuction of subcutaneous abdominal or hip fat was taken and prepared for example using the established PureGraft™ method (Cytori GmbH, Switzerland). This involved centrifugation and concentration in order to remove the tumescent solution. Digestion was then performed for 60 min at 37° C. in 0.15% (w/v) collagenase NB 6 GMP grade from Clostridium histolyticum (0.12 U/mg collagenase; SERVA Electrophoresis GmbH) diluted in phosphate-buffered saline (PBS; Gibco). After centrifugation at 180 g for 10 minutes, the lipid-rich layer was discarded and the cell pellet washed once with PBS. Erythrocytes were then dissolved by 2 minutes' incubation in lysis buffer (0.15 M ammonium chloride, Sigma-Aldrich). The stromal vascular fraction (SVF) obtained was suspended in complete medium (CM, Gibco).

### Production of a Skin Equivalent

The dermis equivalent was built up on the basis of a 2 mm thick collagen/elastin matrix (Matriderm®, Dr. Suwelack Skin & Health Care AG). A piece was punched out using a round punch and transferred into a Transwell®. This was coated on ice with a collagen I solution containing dermal fibroblasts. The silicone plunger with the cylinders was gently rotated. The gel was then polymerized for 15 min, after which it was transferred into a centrifuge. After centrifugation, the water was removed and the cavities formed were coated in succession with ECM and colonized with the hair follicle cells.

1. A method for producing a skin equivalent comprising the steps of:
a) producing a dermal matrix containing a first layer and a second layer comprising the steps of:
i) providing the first layer;
ii) providing a polymerizable solution comprising a liquid and at least one polymer selected from collagen, elastin and/or hyaluron;
iii) adding the polymerizable solution to the first layer;
iv) polymerizing the solution to form the second layer;
v) compressing the second layer, wherein as a result of the compression the liquid content of the second layer is reduced and the first layer and second layer are joined together to form the dermal matrix; and
vi) introducing at least one cavity into the dermal matrix;
b) introducing at least one hair follicle and/or hair follicle germ into the at least one cavity of the dermal matrix produced in step a) to produce a skin surrogate; and
c) adding at least one further cell population selected from fibroblasts, endothelial cells, adipocytes, nerve cells, gland cells, melanocytes or keratinocytes to the skin surrogate to produce the skin equivalent.

2. The method according to claim 1, wherein the first layer comprises a natural skin substance or an artificial skin substance.

3. The method according to claim 1, wherein the first layer contains substantially no living cells.

4. The method according to claim 1, wherein the polymerizable solution additionally contains cells.

5. The method according to claim 1, wherein prior to step iv) the polymer in the polymerizable solution is patterned by agitation.

6. The method according claim 1, wherein compression of the second layer proceeds by centrifugation, sedimentation or mechanical application of force.

7. The method according to claim 1, wherein method steps iv), v) or vi) take place in succession and/or simultaneously.

8. The method according to claim 1, wherein introduction of the at least one cavity into the dermal matrix in step vi) proceeds during or after the polymerization in step iv), during the polymerization.

9. The method according to claim 1, wherein the at least one cavity is introduced into the dermal matrix by piercing, excision or by means of an impression mold.

10. The method according to claim 1, wherein the cavities have an average diameter of ≥0.001 mm to 5 mm.

11. The method according to claim, wherein the cavities have a depth of ≥0.05 mm to 10 mm.

12. The method according to claim 1, wherein one cavity or a plurality of or all of the cavities in the dermal matrix is/are coated with extracellular matrix proteins prior to introduction of the hair follicle and/or hair follicle germ in step b).

13. A skin equivalent produced using the method according to any one of claim 1.

14. A transplant comprising an effective amount of skin equivalent according to claim 13, optionally together with pharmaceutically acceptable adjuvants.

15. A method for in vitro screening of substances with skin- and/or hair-regulating, comprising the steps of:
a) providing a sample of the skin equivalent according to claim 13;
b) dividing the sample into portions;
c) incubating at least one portion with substances which are to be tested; and
d) comparing the parameters of the skin and/or hair in the portion with another portion which has not been incubated with the substances.
